(19) United States
(12) Patent Application Publication
(10) Pub. No.: US 2019/0364921 A1
(43) Pub. Date: Dec. 5, 2019

(54) METHOD FOR MANUFACTURING CORDYCEPS SINENSIS-MYCELIATED GREEN TEA

(71) Applicant: MycoTechnology, Inc., Aurora, CO (US)

(72) Inventors: Brooks John KELLY, Aurora, CO (US); James Patrick LANGAN, Aurora, CO (US)

(21) Appl. No.: 16/544,756

(22) Filed: Aug. 19, 2019

Related U.S. Application Data
(63) Continuation-in-part of application No. 14/734,943, filed on Jun. 9, 2015.
Application No. 14/734,943 is a continuation of application No. 13/844,685, filed on Mar. 15, 2013, now Pat. No. 9,068,171.
(60) Provisional application No. 61/697,506, filed on Sep. 6, 2012.

(51) Int. Cl. A23F 5/02; C12N 1/14; A23L 5/00; A23F 5/20; A23L 31/00

(52) U.S. Cl. CPC A23F 5/02; C12N 1/14; A23L 7/00; A23F 5/204; A23L 31/00; A23L 5/00

(57) ABSTRACT

Provided is a method for production of an improved myceliated green tea with increased herbal/vegetal/hay or floral notes, the method including the steps of providing green tea leaves, providing a fungal aqueous culture with Cordyceps sinensis fungus, inoculating the green tea leaves with at least a portion of the fungal liquid tissue culture; and culturing the fungal mycelium with the green tea leaves to produce the improved myceliated green tea, and teas produced by the methods disclosed.

### CROSS-REFERENCE TO RELATED APPLICATIONS

This patent application is a continuation in part of U.S. application Ser. No. 14/734,943, filed Jun. 9, 2015, which is a continuation of U.S. patent application Ser. No. 13/844,685, filed Mar. 15, 2013, now U.S. Pat. No. 9,068,171, which application claims the benefit of U.S. Provisional Application No. 61/697,506, filed Sep. 6, 2012, each of which are incorporated herein by reference in their entirety.

### BACKGROUND OF THE INVENTION

Camellia sinensis leaves, among other teas, have been used in traditional Chinese medicine to alleviate certain symptoms and improve general health. Examples of these include, but are not limited to, blends with Chrysanthemum indicum, Gingko biloba, Osmanthus fragrans, Ionicera japonica, Gomphrena globosa and Lavendula angustifolia.

Green tea is made from tea leaves that are simply steamed and dried soon after harvest. Black tea is dried and crushed which allows the action of enzymes (which occur naturally in the tea leaves) to convert some of the simple catechins of green tea to more complex forms. Green Tea notes, which range from really vegetal and grassy, to buttery and nutty with hints of alfalfa, persimmon, and hay.

Fermented green teas are also known, although the organism responsible for fermented teas is understood to be Aspergillus niger and/or Aspergillus luchuensis.

It would be desirable to produce a fermented green tea beverage with increased desirable flavors, such as increased floral, vegetal and herbal notes.

### SUMMARY OF THE INVENTION

Accordingly, the present invention provides a method for production of an improved myceliated green tea with increased herbal/vegetal/hay or floral notes, the method including the steps of providing green tea leaves, providing a fungal aqueous culture with Cordyceps sinensis fungus, inoculating the green tea leaves with at least a portion of the fungal liquid tissue culture; and culturing the fungal mycelium with the green tea leaves to produce the improved myceliated green tea, and teas produced by the methods disclosed.

### DETAILED DESCRIPTION OF THE INVENTION

The present invention discloses methods of myceliating green tea leaves with solid state Cordyceps sinensis. The products manufactured according to the methods of the present invention produce a tea leaf that when brewed has increased floral and herbal/vegetal notes. The present inventors have achieved the development of C. sinensis myceliated (hereinafter also referred to as fermented) tea, a new tea product. Simple to produce and process and useful as an every-day drinking tea, C. sinensis tea as disclosed herein is prepared by solid-state fermentation of inexpensive, unfermented Camellia sinensis green tea.

In one embodiment, the method includes a method for production of a myceliated tea. In one embodiment, the tea may be green tea. The steps may comprise, consist of, or consist essentially of the following steps. In one step, a substrate comprising tea leaves are provided. In another step, a fungal liquid tissue culture is provided. In one embodiment, the fungal liquid tissue comprises Cordyceps sinensis and includes fungal biomass. In another step, the substrate comprising tea leaves are inoculated with at least a portion of the fungal liquid tissue culture. The substrate comprising tea leaves can then be cultured (e.g., incubated, or fermented) to result in the myceliated substrate comprising tea. In one embodiment, the myceliated substrate comprising tea has a flavor profile having improved qualities, e.g., increased herbal/grassy/vegetal notes and/or increased floral notes.

In one step, a substrate comprising, optionally, green tea leaves are provided. “Tea leaves” generally refers to leaves from the tea plant Camellia sinensis, which may be blended with other herbal species such as Chrysanthemum indicum, Gingko biloba, Osmanthus fragrans, Ionicera japonica, Gomphrena globosa and Lavendula angustifolia. Green tea is generally considered to be tea leaves which have undergone the least amount of oxidation of any of the generally known tea processing steps. The oxidation process is halted by the quick application of heat after tea picking, either with steam or by dry roasting in hot pans, for example. The leaves are then left to dry, for example, as separate leaves. Typically, the tea leaves are processed within one or two days of harvesting and the chemical composition of the fresh leaves is preserved the most out of the different types of processing tea leaves. The quick application of heat inactivates the enzymes in the tea leaves (also called “fixation”)

The tea leaves may be used in the present invention either before or after fixation. If the tea leaves are to be processed by optional sterilization as described elsewhere herein, the tea leaves may be placed into the methods of the invention in the absence of a fixation step. In such a method, the tea leaves should be entered into the present methods quickly after harvest to minimize oxidation from any sources, or preserved in such a way as to minimize oxidation prior to entry into the methods of the invention.

In other embodiments, tea other than “green tea” are also included in the methods of the present invention. For example, the method may include use of “yellow tea”, which differs from green tea processing in that instead of drying after fixation, the leaves are allowed to further oxidize in a heated humid environment without input from enzymes or microbes. “Oolong” teas, in which oxidation is allowed to occur for some period of time, may also be used.

In one embodiment, the flavor produced by tea leaves myceliated by the present invention has increased vegetal/herbal/grassy notes and increased floral notes. The present invention's tea also modulates bitter aftertastes and astringency, and in embodiments, may reduce both.

Hydration ensures that the tea leaves have optimal moisture content for myceliation. Hydration may be accomplished by methods as known in the art. In the embodiment where the dried tea leaves are placed in an appropriately outfitted food-grade fermenter, the tea need not be hydrated if the relative humidity is kept high enough. This can be determined by one of skill in the art. In one embodiment, described elsewhere herein, an optional sterilization step aids in mildly hydrating the tea leaves.

The hydration may be accomplished by an aqueous medium. The aqueous medium includes water and optionally, additional excipients. Water may be distilled or mineralized. Other excipients can be added to the water, such as buffers to maintain a certain pH, sodium chloride, citric acid and/or ascorbic acid. The pH may be neutral or adjusted. The temperature of the aqueous medium may be room temperature, or elevated in temperature to accelerate the hydration process. Hydration may be accomplished by allowing the green coffee beans to soak in the aqueous medium for any appropriate length of time, ranging from a few seconds or less to overnight. Moisture content of the hydrated tea is optionally between about 20% and about 95% moisture content, or between about 30% and about 70% moisture content, or between about 40% and 50%. In one embodiment, the moisture content is at least about 40%, at least about 50%, or at least about 60%.

The methods of the present invention further optionally comprise a method of heat treatment (e.g. pressurized saturated steam treatment) to effect, in one embodiment, a pasteurization, and in another embodiment, a sterilization of the optionally hydrated provided tea. This step may be accomplished by any method known in the art or by methods disclosed herein. As an example of pasteurization, hydrated tea may be subjected to dry heat treatment at atmospheric pressure at temperatures of about 145 to 190° F. for 30 to 90 minutes, or alternatively at 140 to 210° F. for 20 to 100 minutes. Sterilization may be performed as is known the art. For example, tea may be sterilized by heating under a pressure of 15 lb/in² at 121 to 122° C. for 20 to 150 minutes, such as 120 minutes, depending on size of the batch and conditions of the sterilization. In another embodiment, the steam is superheated to 122 to 125° C. The pressures may vary from 5 to 25 lb/in², depending on the altitude of the processing location. Tea may be sterilized in a container that is sealed or unsealed. In one embodiment, hydrated tea in an autoclavable container is sterilized in a pressure vessel, such as an autoclave (bags can be sterilized on a liquid cycle). In another embodiment, tea is sterilized by injecting saturated steam at pressures and temperatures described above into the food-grade fermenter holding the tea. In this embodiment, the tea should be agitated during the sterilization to ensure even heat treatment for eventual homogenous roasting profiles. Biological tests using Bacillus stearothermophilus can be used to ensure and optimize sterilization cycles.

Suitable containers include containers known in the art for mushroom cultivation. Optionally the containers have a section for exchanging air or gases but do not allow passage of any other component. Such sections are known in the art and include filter strips. In one embodiment, the container is a drum, for example, a 55 gallon drum. Once sterilized, the tea is optionally cooled to approximately 60 to 90° F. before being inoculated. This process can be accomplished by any method known in the art, and hastened through the use of central air temperature control, refrigerators, heat exchangers, or glycerol chillers.

The fungal component of the present invention is a culture comprising C. sinensis. However, it is understood by the inventors that the property of improving taste profile will likely be found in other genii of fungus, and likely other species of Cordyceps fungus, such as, for example, any species of Ophiocordyceps, Elaphocordyceps, or Cordyceps, such as C. sinensis and C. militaris. In one embodiment, the C. sinensis culture is strain WC859 which is commercially available, e.g., from Pennsylvania State University (The Pennsylvania State University Mushroom Culture Collection, available from the College of Agriculture Sciences, Department of Plant Pathology and Environmental Microbiology, 117 Buckhout Laboratory, The Pennsylvania State University, University Park, Pa., USA 16802). This C. sinensis strain was determined by the inventors to efficiently grow on, metabolize, and otherwise utilize, tolerate and modify Camellia sinensis leaves to effectively modify the taste of the beverage brewed from them.

Fungi used in the present invention may be prepared by methods as described herein. For example, in one embodiment, the fungal component is optionally grown, maintained, and/or propagated by methods known in the art. In one embodiment, the media is an undefined organic food medium. In one embodiment, the fungal component is indefinitely maintained in the It is believed that the perpetual and subtle changes made from batch to batch of agar media when using undefined organic food media effectively avoids the phenomenon of undesirable genetic drift that will occur over time to the fungal component when it is maintained on identical iterations of media. The undefined organic food medium comprising may be made by a number of methods. In one embodiment, the undefined medium comprises organic food powder and/or organic fruit puree. Optionally, additional energy sources can be added. Materials are optionally organic and water at least RO filtered. In one embodiment, 14 to 60 g/L of agar is mixed with 2 to 10 g/L organic potato starch powder or potato and 0.2 to 1 g/L organic carrot powder. Aqueous potato mixture can be prepared by softening 1-300 g of potato mass in boiling or pressurized water, mashed, and the filtrate was collected through 1-3 filtrations. Optionally, organic fruit juice or puree can also be added at 0.1 to 10% (v/v). 1.5 L of media can be placed into a 1 gallon jar as such, with water added to clean down the inner and outer walls of the container. In one embodiment, the medium comprises 0.1-10% by weight of malt extract, 0.1-10 by weight undefined vegetable extract, 0.1-10% by weight of yeast extract, 0.1-10% by weight of peptone, 0.1-10% by weight of glucose, 20-80% by weight of water.

Liquid media comprising undefined vegetable powder and fruit juice/puree can be prepared as described for solid media, except that no agar is added. In one embodiment, the fungal component for inoculation into tea leaves can be prepared as a submerged liquid tissue culture using the undefined organic food medium as defined herein and agitated on a shaker table. In one embodiment, the agitation rate is 50 to 240 RPM, or 85 to 95 RPM, and incubated for 1 to 90 days. In one embodiment, the incubation temperature is 87 to 89° F.

In one embodiment the fungal liquid tissue culture component is a fungal strain having an enhanced solid-state myceliation ability to tolerate, grow on and reduce at least one undesirable taste component of green tea, tea plant parts or tea extracts. In another embodiment, the selected strain is capable of efficient solid-state removal of one or more undesirable taste components and is capable of increasing a desirable taste component in green tea plant parts or tea plant extracts. In another embodiment, the methods of the present invention produce a myceliated tea leaf that has increased hay/herbal/vegetal and/or floral notes when brewed.

In one embodiment, methods for preparing the fungal culture to inoculate the tea leaves include scaling up a fungal culture as defined herein in liquid culture. In one embodiment, the fungal culture is in the solid state. In another embodiment, the fungal culture is in the liquid state, either of the floating or submerged morphology. Liquid culture can be accomplished by any means known in the art and includes use of a bioreactor, especially in the one embodiment where the tea leaves are in a food-grade fermenter, wherein an entire bioreactor of culture, for example a 100 L bioreactor, can be used to inoculate large batches of substrate, say, 10,000 lb. For example, when using a bioreactor to prepare the fungal culture, the bioreactor can be prepared by diluting undefined liquid organic food media up to 1000× with RO/distilled water.

The fungal culture may be optionally agitated during culturing by methods known in the art. For example, in a bioreactor, the agitation may be accomplished by a combination of sparged air and a motorized paddle which allows both a turbulent environment and shear mechanical force. The inventors, without limitation, have found that the combination is superior to running either method individually, as sparged air creates the most turbulence at the top half of the culture, while affecting the bottom less, which can be kept agitated by a motorized paddle, while the paddle does not have to run at such a high RPM as normally used in the art. The combination creates the proper small hyphael sphere sizes without damaging the mycelia.

Liquid state fermentation agitation and swirling techniques are known in the art and include mechanical shearing using magnetic stir bars, stainless steel impellers, injection of sterile high-pressure air, and/or the use of shaker tables. Higher agitation and swirling rates, in conjunction with air and media injections, produce smaller mycelial spheres. In some embodiments, the mycelium grows as a floculant culture, depending on the linear combination of agitation methods.

The fungal culture can be grown until ready for inoculation of the tea leaves as determined by one of skill in the art. In some embodiments, the fungal culture can be grown for 1 to 10 days prior to use in inoculating the tea leaves. Determination of whether the fungal cultures are suitable for inoculation of the tea leaves can be determined by one of skill in the art. For example, in one embodiment, the fungal culture, when in liquid media, is suitable for inoculation while in log phase, either early or late. Senescent cultures and cultures in earlier growth phases with lower amounts of mycelia/mL can be used, but are not preferred. The prepared fungal culture optionally appears well grown through in the media, with visible mycelia growing through every mL visible by microscope and unassisted vision.

In order to effect the most efficient myceliation of the tea, the fungal culture has defined hyphael sphere sizes which enables hyphae growth in three dimensions around the spherical conglomeration of the culture of the fungal strain. In one embodiment the hyphael sphere size is less than 10 mm in diameter, less than 2 mm in diameter, less than 1 mm in diameter, less than 100 μm in diameter, less than 10 μm in diameter, less than 5 μm in diameter, or a size range of 10 to 50 μm in diameter. These methods result in a prepared fungal culture for the inoculation of the tea leaves.

The tea leaves, in one step, are inoculated with the fungal culture. The fungal culture to be used can be any fungal component as defined in the instant invention. The inoculation of the fungal culture onto the tea leaves can be carried out by any method known in the art. This step may be variously referred to as the culturing step, the fermentation step, and/or the myceliation step.

The myceliation may take place in a container as described herein. In one embodiment, the myceliation takes place in a food-grade fermenter outfitted for various purposes as discussed herein. As also discussed, a bioreactor on the orders of magnitude of tens to hundreds of liters can be used to inoculate said fermenter. The step of myceliating the tea leaves can take place for between one to 90 days, for between about one to twenty-one days, and in one embodiment, for about two to four days, and at any temperature that precludes contamination and thermal shock, for example, room temperature or below. Multiplication of the mycelium by cytokinesis is carried out by efficiently controlling environmental light, such as by a control model of 40% lighting and 60% dark, and also by controlling sterile airflow and temperature at 86 to 88° F. or 87 to 89° F., or between 12 to 35° C., or between 24 to 32° C. Relative humidity of this culturing, myceliation, and/or fermentation step is controlled between 20 to 99%, and in some embodiments, about 70%.

Expansion of the fungus mycelia is monitored by microscopy, and schedules of growth documented by photography. The longer the incubation period, the greater the production of the mycelium dry weight and the greater the flavor change of the myceliated tea leaves. In some embodiments, a general myceliation time of two to four days can be used. In some embodiments, too much mycelial growth will introduce flavor defects in the resultant myceliated tea by way of emphasizing fungal notes. In some embodiments, the moisture content of the tea in combination with the humidity of the air will prevent vigorous myceliation from occurring. When to harvest the myceliated coffee product may be determined by a number of methods. Harvesting is generally performed with a timing to optimize the taste profile of the myceliated tea according to the taste profile desired. For example, the scent profile of the myceliation culture can be used by the trained person to determine when the culture is ready. Determination of the appearance of the culture may also be done by the trained personnalytical methods of analysis including high performance liquid chromatography (HPLC), mass-spectroscopy, and UV-VIS spectrophotometry may be employed to carry out measurement of biomolecules in order to determine the optimum composition and cultivation conditions and the appropriate time(s) for harvesting the myceliated tea.

In some embodiments, once myceliated, the myceliated tea is optionally rinsed after myceliation. Rinsing may be performed to remove some or all parts of the mycelia and/or other non-tea matter.

In some embodiments, once myceliated, the myceliated tea is optionally dried. Drying can be accomplished by means known in the art for drying tea. For example, myceliated tea may be spread on a dry surface to dry in the ambient. A fan can be used to create a laminar air stream over the myceliated tea. An industrial tea dryer can be used. In one embodiment, the myceliated tea is dried down to about an 11 to 13% moisture content.

In another embodiment, the myceliated tea may be dried for 1 to 2 days at 40 to 50° C. Optionally, either in addition to or as an alternative to drying, the myceliated tea may be pasteurized by methods known in the art, for example, by incubation for 2 hours at 60 to 80° C. The myceliated tea may be mixed with other beverages or foods by any methods known in the art, or may be packaged without any other excipients by, for example, vacuum packaging.

The myceliated tea, or products incorporating the myceliated tea, may be brewed by methods known in the art to prepare a beverage for use in food and/or drink products.

The present invention also relates to compositions comprising combinations of a C. sinensis mycelial liquid tissue culture with tea leaves. The mycelial tissue culture and tea leaf containing-compositions, in one embodiment have a taste with improved grassy/herbal/vegetal notes and/or floral notes. The compositions comprising the combinations may be used in food products or beverages that have enhanced tastes relative to the food product or beverage alone, in one embodiment, with improved grassy/herbal/vegetal notes and/or floral notes. In one embodiment, the present invention includes myceliating Camellia sinensis leaves with, for example, Cordyceps sinensis to provide a better tasting Camellia sinensis tea compared to the unmyceliated control.

The following examples further illustrate the invention but, of course, should not be construed as in any way limiting its scope.

### EXAMPLES

### Example 1

C. sinensis was grown by inoculating a petri plate comprising a solid nutrient agar medium (per L:36 g Agar, 21 g Mango Concentrate, 10 grams Corn Powder) with a pure culture of Cordyceps sinensis and incubating the plate at 23 to 26° C. for 6 to 8 days to develop a solid-state inoculant comprising the filamentous form of Cordyceps sinensis.

A single seed culture comprising liquid medium was inoculated with a portion of the inoculated plate from and incubated for 4 to 8 days at 26° C. in a sterile human food-grade liquid medium composed of 8 g/L potato powder and 0.8 g/L carrot powder in RO water. This step was to develop liquid-state inoculant comprising both filamentous and yeast forms of Cordyceps sinensis.

This C. sinensis culture was used to inoculate a solid fermentation medium consisting of pasteurized (e.g. 30 to 60 minutes at 25 lb/in² pressure and 120-121° C.), un-fermented green tea. To inoculate the green tea, 50 mL C. sinensis grown in the aqueous carrot powder and potato starch to about 3-5 g biomass/ml was mixed into 113.5 g of solid, pasteurized green tea in 35.125 mL sterile water, for a total mass of 198.625 g with a moisture level of 42/9%, and incubated at 26° C. for 2-4 days. A control had a sham fermentation.

### Example 2

Tea myceliated with C. sinensis, served hot or cold.

3 teaspoons of each tea were filled with 1000 mL of filtered water, using Breville Tea Maker on Strong setting. All samples were either stored in a hot water bath until served (“served hot”) or cooled in a freezer until 4° C. (“served cold”). Green tea (untreated by the processes of the invention) were also tested served hot or served cold.

Green tea served hot has an upfront floral, musty, earthy note with topcoat. Predominant flavors are musty, earthy, floral, bitter and astringent, with a back-end of grassy, bitter, hay and astringency linger. Myceliated tea served hot has upfront sweet, floral, bitter and hay notes, with a predominant floral, grassy and astringent and backend of grassy, earthy, floral and musty notes with astringent linger. Overall, myceliated tea served hot has greater floral and grassy notes (with improved grassy/herbal/vegetal notes and/or floral notes) and astringency linger than green tea served hot.

Green tea served cold has a hay, bitter, grassy, sweet note with a predominant hat and bitter note with some grassy notes. On the back end, green tea served cold has hay and grassy notes with lingering of hay, grassy, earthy and bitter. Myceliated tea served cold has upfront bitter, sweet, floral, and hay notes with some astringency, with a predominate bitter, floral, hay note and astringent linger. The backend has hay, grassy and bitter notes with an astringent linger. Overall, myceliated tea served cold has some floral and hay notes with bitterness with hay backend and astringency, with improved grassy/herbal/vegetal notes and/or floral notes and green tea served cold has hay, bitter and some grass notes.

On a liking scale, myceliated tea served hot scored the highest in liking over green tea served hot, green tea served cold, and myceliated tea served cold.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for production of a myceliated green tea, comprising:
providing green tea leaves;
providing a fungal liquid tissue culture comprising Cordyceps sinensis;
inoculating the green tea leaves with at least a portion of the fungal liquid tissue culture; and
culturing the fungal mycelium with the green tea leaves to prepare the myceliated green tea, wherein the myceliated green tea has improved flavor profile over untreated green tea.

2. A method according to claim 1 wherein the method further comprises sterilizing the green tea leaves prior to the inoculating step.

3. The method of claim 1, wherein the culturing step occurs at a moisture level of between 30% and 50% w/w.

4. The method of claim 1, wherein the fungal liquid tissue culture is prepared by a method comprising the steps of:
(a) inoculating a petri plate comprising a solid nutrient agar medium with a pure culture of C. sinensis;
(b) incubating the plate at 23 to 26° C. for 6 to 8 days;
(c) inoculating a liquid medium with C. sinensis grown on the incubated plate of step (b); and (d) incubating the liquid culture for 4 to 8 days at 26° C. wherein both filamentous and yeast forms of Cordyceps sinensis are produced.

6. The method of claim 1, wherein the method further comprises drying the myceliated green tea leaves for 1 to 2 days at 40 to 50° C.

7. The method of claim 6, wherein the method further comprises pasteurizing the dried, myceliated green tea leaves for 2 hours at 60 to 80° C.

8. The method of claim 1, wherein the improved flavor profile comprises increased floral and grassy notes.

9. The method of claim 1, wherein the culturing step with the fungal mycelium and the green tea leaves to prepare the myceliated green tea is carried out for between about 2 days and 4 days.

10. The method of claim 1, wherein the green tea leaves comprise Camellia sinensis.

11. The method of claim 10, wherein the green tea leaves further comprise Chrysanthemum indicum, Gingko biloba, Osmanthus fragrans, Ionicera japonica, Gomphrena globosa and/or Lavendula angustifolia.

12. A myceliated green tea product prepared by the method of claim 1.
